SEQUENCE LISTING (19 sequences)

SEQ ID NO: 1 - DNA, 25 bp, Artificial Sequence
  HOXA7 forward primer
cgacgtttacggtaatttgttttgc

SEQ ID NO: 2 - DNA, 19 bp, Artificial Sequence
  HOXA7 reverse primer
tcaaccgcgccatacaacg

SEQ ID NO: 3 - DNA, 29 bp, Artificial Sequence
  HOXA7 probe
tcgttaaaggcgtttgcgataagacggac

SEQ ID NO: 4 - DNA, 21 bp, Artificial Sequence
  SCT forward primer1
agggttcggcgatatttagac

SEQ ID NO: 5 - DNA, 18 bp, Artificial Sequence
  SCT reverse primer1
aacaaccgctaaaaccgc

SEQ ID NO: 6 - DNA, 28 bp, Artificial Sequence
  SCT probe1
ccctcccgcaaacgactaaactcgctaa

SEQ ID NO: 7 - DNA, 20 bp, Artificial Sequence
  SCT forward primer2
tcgttataaaggggttttgc

SEQ ID NO: 8 - DNA, 19 bp, Artificial Sequence
  SCT reverse primer2
cctaacgaacgactcacct

SEQ ID NO: 9 - DNA, 27 bp, Artificial Sequence
  SCT probe2
ttcggggtcgtggtcgtagcgtttagt

SEQ ID NO: 10 - DNA, 22 bp, Artificial Sequence
  SHOX2 forward primer
gttttttggatagttaggtaat

SEQ ID NO: 11 - DNA, 20 bp, Artificial Sequence
  SHOX2 reverse primer
taacccgacttaaacgacga

SEQ ID NO: 12 - DNA, 18 bp, Artificial Sequence
  SHOX2 probe
ctcgtacgaccccgatcg

SEQ ID NO: 13 - DNA, 21 bp, Artificial Sequence
  HOXA9 forward primer
cgggcgtttttcgttttaggc

SEQ ID NO: 14 - DNA, 22 bp, Artificial Sequence
  HOXA9 reverse primer
aaatccgtcccaaacgaaaccg

SEQ ID NO: 15 - DNA, 18 bp, Artificial Sequence
  HOXA9 probe
taaatccccacaactacc

SEQ ID NO: 16 - DNA, 500 bp, Homo sapiens
gtgaacgcgctttttagcaaatatacggcgggggcttctctgttccaaaatgccgagccgacttcttgctcctttgctcccaactcacagagaagcggctacggggcgggcgccggcgccttcgcctcgaccgttccgggcttatacaatgtcaacagccccctttatcagagcccctttgcgtccggctacggcctgggcgccgacgcctacggcaacctgccctgcgcctcctacgaccaaaacatccccgggctctgcagtgacctcgccaaaggcgcctgcgacaagacggacgagggcgcgctgcatggcgcggctgaggccaatttccgcatctacccctggatgcggtcttcaggtaggcgcagtcgctaggcgggccaggctggcggagcgggaccgggagcggggagcgcagcgctggggagcgcggagcgcggggcgcggggccggaagagcggagccaggctgttgcgagccggtagccccgtgactcc

SEQ ID NO: 17 - DNA, 1002 bp, Homo sapiens
acggatggtggggatggatgcaccgaatgatcctgaatcgcgcacttcaaagcagataaaatggcaacaccggccccccaacagtcacgccacgctgaagctggagacgaactcgccccagcaaggtgctccaggggcctccggaactccggccgcagccccttccccggggccccccagtccccccccaaaccctcccccaagccctccggcccctccgcccccgcaggctccgaggcttcgcggtgcggttccctctgcgggtcccaggcccgcgctccgttcgccccccaggccgggacgtccccctcccccagccggccgaggggcgccagcgcggcggagggggcgggaggccgggccggggcgacagctgggggggcggccctgaccttcccgggatcgctgggcgcggccgggccgtcccctttccgggcgccgccataaaggggccttgcgggggcccggggccgtggccgcagcgctcagctcctgcgccccgaccccgccatggccccccggcccctcctgctgctgctgctgctcctcgggggctccgccgcgcgccccgcgccccccaggtgagccgcccgccaggcccgcccgccccccgggccccacccgcccccagtgcgccctgaccccgacccccgaccgcccgcagggcccggcgacactcagacgggacgttcaccagcgagctcagccgcctgcgggagggcgcgcggctccagcggctgctacagggcctggtggggaagcgcaggtgaggccgggtggggtggggggtcctggcgtggtgccccagcagcaacgcgcgaccccccagctccaggactaactctgttttggggcggggggcagcgagcaggacgcagagaacagcatggcctggaccaggctcagcgcgggtctgctctgcccgtcagggtccaacatgcccatcctgcaggcctggtgagcacccgccccaccgcagaccccactccagaccccacccca

SEQ ID NO: 18 - DNA, 1002 bp, Homo sapiens
cagcactcacagaaatcaaaggcgcagcgccaggcctctcacagagcaaagtctggacataagggcgcggcaaggccacccactggcctctcacaccggccattcccagaaggctggctcgttctcaggccagctctcaccaccgctggctctctgcctaccgcaaacttgctggtctaatttaggaacaattgggccgaaaggtatcagcgagagcaacagaccccggtgttgtgccgcacagggagccgcatccgcagacgcccctcgctgcccctgggctcgggccaaaccctgcataaggtcccctggacagccaggtaatctccgtcccgcctgcccgaccggggtcgcacgagcacaggcgcccacgccatgttggctgcccaaagggctcgccgcccaagccgggccagaaggcaggaggcggaaaaccagcctccggtggcgggcgaaagcaaccgctctttctgttctctcttcgccctccctcgtggaaacgcagactcgaccctaaacgcttaacccacagagatcaacaggttcaagcggaatattcgcgatcctcggtttctattggttgctcaaagccttttcatgcaaccagcagctcggatgtttaataaaatatgaatttttctgctctgggtttgcacacatccgttgccactttcctgtccttctcctccctttaaaaacaggagctcttctagtgtgagcatatacagccaccatatatatatccccctggaacctttatcctctgcgcccgaatcagagactccgggttttaagaagtcaaagctgagtttagatagtcgaggctgtttgcagttcttcccagcgcaggatccaggccagcatgggtgttaaaaatcatttccggaagagccggtctgcagagaaattaatacttgccctgagcttggaaatcaacaatcccaggaactgcgcccgccgggggttcagacctgaacctcgccgtcaggcgcgctggcgaac

SEQ ID NO: 19 - DNA, 700 bp, Homo sapiens
gctgggattcacggagcgcaaccaggcttgcagcgctcatggttagagcctctgaggctggagcacagggctgggtcgccagccgcctgcgcctgggaatcctgattgccagctgatgagaaaggcgggctgggcgcgcgtgtgcgtggggtcgagggccggggaccgagcgcgccgcacaaccaaccaggccctcaaaaccttcgccctggtggcggctggccgctccctcctggccagctcctccgtggggtcctcgtagcaaaggcgaatttaagggttgcccgggcgcccctcgctccaggcgggtagctgtggggacctacacccgcggtactccctgagcggccggtccctgcctggagtgccctggtagggccggcggcggctccgtttgggacggatcctgcgttgaatttgacttttcgagggcggccgcgggtaaactcgcctctcccggggaccgcagggattatttacagggagctcgccaaccaaacacaacagtctaacctttccaagtcctcgtaaatttttacagctgggagccacggcgaggcaaacgaatctgttggtcgtttccgacttcccgccagcctgtgtggcttctgaaacaataactccttatgaaatatcataaatatagatttaaatacagtagagcgacaatgcgatttggctgcttttttatggcttcaat

(19) United States
(12) Patent Application Publication
(10) Pub. No.: US 2022/0170112 A1
(43) Pub. Date: Jun. 2, 2022

(54) METHODS AND COMPOSITIONS FOR LUNG CANCER DETECTION

(71) Applicant: SUZHOU HOHO HEALTH CO., LTD., Suzhou, Jiangsu (CN)

(72) Inventors: Dan LI, Jiangsu (CN); Nan JIANG, Jiangsu (CN); Daixing LIU, Jiangsu (CN)

(21) Appl. No.: 17/618,239

(22) PCT Filed: Jul. 6, 2019
(86) PCT No.: PCT/CN2019/094956

§ 371 Date: Dec. 10, 2021

(51) Int. Cl. C12Q 1/6886; C12Q 1/6806; C12Q 1/686

(52) U.S. Cl. CPC C12Q 1/6886; C12Q 1/6806; C12Q 2600/16; C12Q 2600/154; C12Q 1/686

(57) ABSTRACT

Provided are methods of diagnosing lung cancer in a human, comprising the steps of: (a) isolating genomic DNA from a bronchial washing sample or bronchial brush from a human, detecting the methylation status of a plurality of genes consisting of HOXA9, SHOX2, SCT and HOXA7 are present in said genomic DNA, wherein each of said gene comprises at least one CpG island which may be methylated, wherein the presence of a hypermethylation status of at least two of the genes is indicative of the presence of lung cancer in said human.

### CROSS-REFERENCE TO RELATED APPLICATION

This application is a U.S. national phase application of International Application No. PCT/CN2019/094956, filed Jul. 6, 2019, the entire disclosure of which is incorporated herein by reference in its entirety for all purposes.

### SEQUENCE STATEMENT

The Sequence listing for this application is labeled “55566_00002_SL.txt” which was created on Jul. 5, 2019 and is 7.43 KB. The entire content of the sequence listing is incorporated herein by reference in its entirety.

### FIELD OF THE INVENTION

The present invention relates to the use of a panel of DNA methylation biomarkers for the detection and diagnosis of lung cancer. Specifically, the present invention provides a method of detecting methylation status of a panel of genes and using them to predict or diagnose lung cancer in humans.

### BACKGROUND OF THE INVENTION

Lung cancer is the leading cause of cancer deaths worldwide (Siegel R L et al., (2019) Cancer statistics. CA Cancer J Clin 2019; 69:7-34). As with most cancers, early detection as well as regular monitoring is critical for good prognosis and highly recommended, ideally using non-invasive diagnostic techniques. Yet biopsy is often needed when patients are suspected of lung cancer, and its sensitivity is often less than satisfactory. Bronchoscopy, transthoracic needle, and surgical lung biopsies are the three most common biopsy procedures.

Among these three procedures, bronchoscopy is relatively safe with fewer complications such as pneumothorax (Tukey M H, Wiener R S. (2012) Population based estimates of transbronchial lung biopsy utilization and complications. Respir Med. 106: 1559-65). However, the sensitivity of bronchoscopy is limited, ranging from 70-80% even with the most advanced navigation systems (Wang Memoli J S. et al., (2012) Meta-analysis of guided bronchoscopy for the evaluation of the pulmonary nodule. Chest. 142: 385-93)—it is also highly dependent on the size and location of the lesion (Rivera M P et al., (2013) Establishing the diagnosis of lung cancer: diagnosis and management of lung cancer, 3rd ed: American College of Chest Physicians evidence-based clinical practice guidelines. Chest. 143(Suppl 5): e142S -e165S). In fact, nondiagnostic bronchoscopic examination remains a major problem in lung cancer diagnosis. Two major factors cause bronchoscopic nondiagnosis: firstly bronchoscopy is not able to reach all lesions especially peripheral lesions even with an advanced navigation system; secondly navigation system does not guide bronchoscopy to lesions accurately.

Many types of body fluids have been proposed for lung cancer detection including blood, sputum, bronchoalveolar lavage (Doseeva V et al., (2015) Performance of a multiplexed dual analyte immunoassay for the early detection of non-small cell lung cancer. J Transl Med. 13:55; Aravanis A M et al., (2017) Next-generation sequencing of circulating tumor DNA for early cancer detection. Cell. 168:571-574; Xing L et al., (2015) Sputum microRNA biomarkers for identifying lung cancer in indeterminate solitary pulmonary nodules. Clin Cancer Res. 21:484-489; Gumireddy K et al., (2015) AKAP4 is a circulating biomarker for non-small cell lung cancer. Oncotarget 6:17637-17647; Zhang C et al., (2017) DNA methylation analysis of the SHOX2 and RASSF1A panel in bronchoalveolar lavage fluid for lung cancer diagnosis. J Cancer. 8:3585-3591). Various molecules have been studied to serve as biomarkers for lung cancer including microRNAs, DNA mutation, and gene expression profiling (Sozzi G et al., (2014) Clinical utility of a plasma-based miRNA signature classifier within computed tomography lung cancer screening: a correlative MILD trial study. J Clin Oncol. 32:768-773; Phallen J et al., (2017) Direct detection of early-stage cancers using circulating tumor DNA. Sci Transl Med. 9:eaan2415; Weiss G et al., (2017) Validation of the SHOX2/PTGER4 DNA methylation marker panel for plasma-based discrimination between patients with malignant and nonmalignant lung disease. J Thorac Oncol. 12:77-84; Showe M K et al., (2009) Gene expression profiles in peripheral blood mononuclear cells can distinguish patients with non-small cell lung cancer from patients with nonmalignant lung disease. Cancer Res. 69:9202-9210; Spira A et al., (2009) Airway epithelial gene expression in the diagnostic evaluation of smokers with suspect lung cancer. Nat Med. 13:361-366).

DNA methylation of multiple genes may contribute to the pathogenesis of lung cancer, and DNA methylation of many genes has been reported in different lung cancer patients. Differential DNA methylation profiles have therefore been found to distinguish lung cancer from normal lung tissues (Y. Goto et al., (2009) Epigenetic profiles distinguish malignant pleural mesothelioma from lung adenocarcinoma. Cancer Res. 69:9073-9082). Multiple methylation panels have even been proposed to serve as biomarkers for lung cancer detection (Xing L et al., (2015) supra; Zhang C et al., (2017) supra; Weiss G et al., (2017) supra; Diaz-Lagares A et al., (2016) A novel epigenetic signature for early diagnosis in lung cancer. Clin Cancer Res. 22:3361-3371; Ooki A et al., (2017) A panel of novel detection and prognostic methylated DNA markers in primary non-small cell lung cancer and serum DNA. Clin Cancer Res. 23:7141-7152).

One of the genes that exhibit DNA methylation associated with lung cancer is HOXA7, a member of the homeobox gene family. HOXA7 methylation was found to serve as a biomarker for lung cancer detection in plasma and sputum (Hulbert A et al., (2016) Early detection of lung cancer using DNA promoter hypermethylation in plasma and sputum. Clin Cancer Res. 23:1998-2005). Another member of the homeobox gene family is HOXA9, and its aberrant methylation was found in lung cancer tissues when compared to normal lung tissues and in plasma and sputum (Sandoval J et al., (2013) A prognostic DNA methylation signature for stage I non-small-cell lung cancer. J Clin Oncol. 31:4140-4147; Nawaz I et al., (2014) Development of a multiplex methylation specific PCR suitable for early detection of non-small cell lung cancer. Epigenetics. 9:1138-1148; Lissa D et al., (2018) HOXA9 methylation and blood vessel invasion in FFPE tissues for prognostic stratification of stage I lung adenocarcinoma patients. Lung cancer. 122:151-159; Robles A I et al., (2015) An integrated prognostic classifier for stage I lung adenocarcinoma based on mRNA, microRNA, and DNA methylation biomarkers. J Thorac Oncol. 10:1037-1048).

The methylation of another homeo-domain transcription factor, short statue homeobox gene 2 (SHOX2), was shown to serve as a biomarker to distinguish lung cancer from normal lung or benign lung diseases. Its methylation status in plasma, pleural effusion, and bronchial aspirates has been found to differentiate lung cancer from benign lung diseases (Ilse P et al., (2013) Shox2 DNA methylation is a tumour marker in pleural effusions. Cancer Genomics & Proteomics. 10:217-224; Schmidt B et al., (2010) SHOX2 DNA methylation is a biomarker for the diagnosis of lung cancer based on bronchial aspirates. BMC Cancer. 10:600; Kneip C et al., (2016) SHOX2 DNA is a biomarker for the diagnosis of lung cancer in plasma. J Thorac Oncol. 6:1632-1638).

Secretin (SCT) was also found to be hypermethylated in lung cancer tissues and may serve as a potential biomarker for lung cancer and other types of solid tumor (Zhang Y et al., (2015) Validation of SCT methylation as a hallmark biomarker for lung cancers. J Thorac Oncol. 11:346-360).

The prior art methods of lung cancer detection based on DNA hypermethylation, however, has not been satisfactory, due to their relatively low sensitivity, and/or need for an invasive step (e.g. taking a blood sample from the patient). Further, it has not shown that the combination of a limited number of hypermethylation sites can be used with non-invasive sampling, i.e., bronchial washing and brush, to overcome the shortcomings of the prior art methods.

Thus, there exists a continuing need in searching for a panel of genes that exhibit DNA methylation associated with lung cancer. There is also a need to utilize a non-invasive test to detect DNA methylation of such gene panel in order to serve as biomarkers in diagnosing and monitoring an increased risk (in occurrence) of lung cancer.

### SUMMARY OF THE INVENTION

The present inventors have successfully identified the DNA methylation of a panel of genes as biomarkers of lung cancer.

In one aspect, the present invention provides a method of detecting lung cancer in a patient, comprising the steps of obtaining a bronchial washing sample, or a bronchial brush sample from the patient, isolating genomic DNA from said sample, conducting at least one round of PCR on said sample, in such a way to determine if the known CpG methylation sites on the genes were hypermethylated, and determining that the patient is suffering from lung cancer if at two of the genes are methylated in said sites.

In particular, the present inventors have discovered that a plurality of genes, e.g. the HOXA9, SHOX2, SCT and HOXA7 genes, each contains at least one CpG island which may be methylated, and the hypermethylation status is indicative of lung cancer in a human patient.

Thus the present invention provides a method for lung cancer detection by determining the hypermethylation status of the CpG islands of these genes. One of ordinary skills in the art recognizes that the hypermethylation status of a gene can be determined in many ways, using widely known and well-established method, such as conventional PCR, quantitative real time PCR or high-throughput sequencing. For example, a method may comprise the following steps: (i) converting unmethylated cytosine to uracil in the genomic DNA using sodium bisulfite; (c) performing a round of PCR on said sodium bisulfite-treated genomic DNA to amplify, wherein the PCR primers are designed in such a way that if the CpG islands are methylated, successful amplification will occur and a PCR product can be detected, while on the other hand, if there is no methylation, the PCR primers will not anneal to the site and no PCR amplification will result.

In a preferred embodiment, the presence of a hypermethylation status of at least two of the genes indicates with particularly high sensitivity and specificity as an indication of the presence of lung cancer in said human.

In a preferred embodiment, the presence of a hypermethylation status of three of the genes indicates with particularly high sensitivity and specificity as an indication of the presence of lung cancer in said human.

In a preferred embodiment, the presence of a hypermethylation status of four of the genes indicates with particularly high sensitivity and specificity as an indication of the presence of lung cancer in said human.

In one embodiment, the present disclosure provides a method of diagnosing lung cancer in a human subject, comprising the steps of: (a) isolating genomic DNA from a bronchial washing sample or bronchial brush from a human, wherein a plurality of genes consisting of HOXA9, SHOX2, SCT and HOXA7 are present in said genomic DNA; (b) converting unmethylated cytosine to uracil in the genomic DNA using sodium bisulfite; (c) performing a round of PCR on said sodium bisulfite-treated genomic DNA to amplify at least two, such as two, three or four, of the four genes; and (d) detecting the presence or absence of PCR amplification result; wherein the presence of PCR amplification results in one of the genes indicates the presence of a hypermethylation status of the gene, and wherein the presence of a hypermethylation status of at least one of the genes is indicative of the presence of lung cancer in said human. The pair of primers for amplifying the four genes are as follows:

| | HOXA7 forward primer: |
|---|---|
| (SEQ ID NO.: 1) | |
| | CGACGTTTACGGTAATTTGTTTTGC |
| | HOXA7 reverse primer: |
| (SEQ ID NO.: 2) | |
| | TCAACCGCGCCATACAACG |
| | SCT forward primer1: |
| (SEQ ID NO.: 4) | |
| | AGGGTTCGGCGATATTTAGAC |
| | SCT reverse primer1: |
| (SEQ ID NO.: 5) | |
| | AACAACCGCTAAAACCGC |
| | SCT forward primer2: |
| (SEQ ID NO.: 7) | |
| | TCGTTATAAAGGGGTTTTGC |
| | SCT reverse primer2: |
| (SEQ ID NO.: 8) | |
| | CCTAACGAACGACTCACCT |
| | SHOX2 forward primer: |
| (SEQ ID NO.: 10) | |
| | GTTTTTTGGATAGTTAGGTAAT |
| | SHOX2 reverse primer: |
| (SEQ ID NO.: 11) | |
| | TAACCCGACTTAAACGACGA |
| | HOXA9 forward primer: |
| (SEQ ID NO.: 13) | |
| | CGGGCGTTTTTCGTTTTAGGC |
| | HOXA9 reverse primer: |
| (SEQ ID NO.: 14) | |
| | AAATCCGTCCCAAACGAAACCG. |

In an embodiment, the following probes may be used in gene detection:

| | HOXA7 probe: |
|---|---|
| (SEQ ID NO.: 3) | |
| | TCGTTAAAGGCGTTTGCGATAAGACGGAC |
| | SCT probe1: |
| (SEQ ID NO. :6) | |
| | CCCTCCCGCAAACGACTAAACTCGCTAA |
| | SCT probe2: |
| (SEQ ID NO.: 9) | |
| | TTCGGGGTCGTGGTCGTAGCGTTTAGT |
| | SHOX2 probe: |
| (SEQ ID NO.: 12) | |
| | CTCGTACGACCCCGATCG |
| | HOXA9 probe: |
| (SEQ ID NO.: 15) | |
| | TAAATCCCCACAACTACC. |

In another embodiment, the present disclosure provides a method of diagnosing lung cancer in a human, comprising the steps of: (a) isolating genomic DNA from a bronchial washing sample or bronchial brush from a human, wherein a plurality of genes consisting of HOXA9, SHOX2, SCT and HOXA7 are present in said genomic DNA; (b) converting unmethylated cytosine to uracil in the genomic DNA using sodium bisulfite; (c) performing a round of PCR on said sodium bisulfite-treated genomic DNA to amplify, said round of PCR is performed using a forward primer and a reverse primer for SCT and HOXA7 respectively, said forward primers consisting of the nucleotide sequence of SEQ ID NO:1 (for HOXA7) and SEQ ID NO: 4 (for SCT), and said reverse primers consisting of the nucleotide sequence of SEQ ID NO:2 (for HOXA7) and SEQ ID NO: 5 (for SCT); and (d) detecting the presence or absence of PCR amplification result; wherein the presence of a PCR amplification results in one of the genes indicates the presence of a hypermethylation status of the gene, and wherein the presence of a hypermethylation status of both of the genes is indicative of the presence of lung cancer in said human. In step (c), the forward primer and reverse primer for SCT can be alternatively SEQ ID NO.: 7 and 8, respectively.

In another embodiment, in step c) above, the round of PCR is performed on said sodium bisulfate-treated genomic DNA using a forward primer and a reverse primer for SHOX2, SCT and HOXA7 respectively, said forward primers consisting of the nucleotide sequence of SEQ ID NO:1 (for HOXA7), SEQ ID NO: 4 (for SCT) and SEQ ID NO: 10 (for SHOX2), and said reverse primers consisting of the nucleotide sequence of SEQ ID NO:2 (for HOXA7), SEQ ID NO: 5 (for SCT) and SEQ ID NO: 11 (for SHOX2); and wherein the presence of a PCR amplification result in one of the genes indicates the presence of a hypermethylation status of the gene, and wherein the presence of a hypermethylation status of all of the genes is indicative of the presence of lung cancer in said human. In step (c), the forward primer and reverse primer for SCT can be alternatively SEQ ID NO.: 7 and 8, respectively.

In another embodiment, in step c), the round of PCR is performed on said sodium bisulfate-treated genomic DNA using a forward primer and a reverse primer for HOXA9, SCT and HOXA7 respectively, said forward primers consisting of the nucleotide sequence of SEQ ID NO:1 (for HOXA7), SEQ ID NO: 4 (for SCT) and SEQ ID NO: 13 (for HOXA9), and said reverse primers consisting of the nucleotide sequence of SEQ ID NO:2 (for HOXA7), SEQ ID NO: 5 (for SCT) and SEQ ID NO: 14 (for HOXA9); and wherein the presence of a PCR amplification result in one of the genes indicates the presence of a hypermethylation status of the gene, and wherein the presence of a hypermethylation status of all of the genes is indicative of the presence of lung cancer in said human. In step (c), the forward primer and reverse primer for SCT can be alternatively SEQ ID NO.: 7 and 8, respectively.

In another embodiment, the method of diagnosing lung cancer in a human of the present invention comprises the steps of: (a) isolating genomic DNA from a bronchial washing sample or bronchial brush from a human, wherein a plurality of genes consisting of HOXA9, SHOX2, SCT and HOXA7 are present in said genomic DNA; (b) converting unmethylated cytosine to uracil in the genomic DNA using sodium bisulfate; (c) performing a round of PCR on said sodium bisulfite-treated genomic DNA to amplify, said round PCR is performed using a forward primer and a reverse primer for HOXA9 and HOXA7 respectively, said forward primers consisting of the nucleotide sequence of SEQ ID NO:1 (for HOXA7) and SEQ ID NO: 13 (for HOXA9), and said reverse primers consisting of the nucleotide sequence of SEQ ID NO:2 (for HOXA7) and SEQ ID NO: 14 (for HOXA9); and (d) detecting the presence or absence of PCR amplification result; wherein the presence of a PCR amplification result in one of the genes indicates the presence of a hypermethylation status of the gene, and wherein the presence of a hypermethylation status of both of the genes is indicative of the presence of lung cancer in said human.

In another embodiment, the method of diagnosing lung cancer in a human of the present invention comprises the steps of: (a) isolating genomic DNA from a bronchial washing sample or bronchial brush from a human, wherein a plurality of genes consisting of HOXA9, SHOX2, SCT and HOXA7 are present in said genomic DNA; (b) converting unmethylated cytosine to uracil in the genomic DNA using sodium bisulfite; (c) performing a round of PCR on said sodium bisulfite-treated genomic DNA to amplify, said round PCR is performed using a forward primer and a reverse primer for SHOX2 and HOXA7 respectively, said forward primers consisting of the nucleotide sequence of SEQ ID NO:1 (for HOXA7) and SEQ ID NO: 10 (for SHOX2), and said reverse primers consisting of the nucleotide sequence of SEQ ID NO:2 (for HOXA7) and SEQ ID NO: 11 (for SHOX2); and (d) detecting the presence or absence of PCR amplification result; wherein the presence of a PCR amplification result in one of the genes indicates the presence of a hypermethylation status of the gene, and wherein the presence of a hypermethylation status of both of the genes is indicative of the presence of lung cancer in said human.

In another embodiment, the method of diagnosing lung cancer in a human of the present invention comprises the steps of: (a) isolating genomic DNA from a bronchial washing sample or bronchial brush from a human, wherein a plurality of genes consisting of HOXA9, SHOX2, SCT and HOXA7 are present in said genomic DNA; (b) converting unmethylated cytosine to uracil in the genomic DNA using sodium bisulfite; (c) performing a round PCR on said sodium bisulfite-treated genomic DNA to amplify, said round PCR is performed using a forward primer and a reverse primer for HOXA9, SHOX2, and SCT respectively, said forward primers consisting of the nucleotide sequence of SEQ ID NO:4 (for SCT), SEQ ID NO:10 (for SHOX2) and SEQ ID NO:13 (for HOXA9), said reverse primers consisting of the nucleotide sequence of SEQ ID NO:5 (for SCT), SEQ ID NO:11 (for SHOX2) and SEQ ID NO:14 (for HOXA9); and (d) detecting the presence or absence of PCR amplification result; wherein the presence of a PCR amplification result in one of the genes indicates the presence of a hypermethylation status of the gene, and wherein the presence of a hypermethylation status of all of the genes is indicative of the presence of lung cancer in said human. In step (c), the forward primer and reverse primer for SCT can be alternatively SEQ ID NO.: 7 and 8, respectively.

The method of the present invention achieves superior results wherein using at least two of the panel of genes, the sensitivity and specificity may even reach as high as 86.4% and 100% respectively.

The present disclosure further provides a method of diagnosing lung cancer in a human, comprising the steps of: (a) isolating genomic DNA from a bronchial washing sample or bronchial brush from a human, wherein a plurality of genes consisting of HOXA9, SHOX2, SCT and HOXA7 are present in said genomic DNA; wherein the HOXA7 gene comprises a sequence of SEQ ID NO: 16, the SCT gene comprises a sequence of SEQ ID NO: 17, the SHOX2 gene comprises a sequence of SEQ ID NO: 18, and the HOXA9 gene comprises a sequence of SEQ ID NO: 19, and wherein each of said gene comprises at least one CpG island which may be methylated, (b) converting unmethylated cytosine to uracil in the genomic DNA using sodium bisulfite; (c) performing a round of PCR on said sodium bisulfite-treated genomic DNA to amplify, wherein said round PCR is performed to determine if said CpG island in said gene is methylated, and wherein the presence of a hypermethylation status of at least two of the genes is indicative of the presence of lung cancer in said human. In one embodiment, a hypermethylation status of at least three of the genes is indicative of the presence of lung cancer in said human. In another embodiment, a hypermethylation status of the HOXA7 and SCT genes is indicative of the presence of lung cancer in said human. In another embodiment, a hypermethlation status of the SHOX2, HOXA7 and SCT genes is indicative of the presence of lung cancer in said human. In another embodiment, a hypermethylation status of the HOXA9, HOXA7 and SCT genes is indicative of the presence of lung cancer in said human. In another embodiment, a hypermethylation status of the SHOX2 and SCT genes is indicative of the presence of lung cancer in said human. In another embodiment, a hypermethylation status of the SHOX2 and HOXA7 genes is indicative of the presence of lung cancer in said human. In another embodiment, a hypermethylation status of the HOXA7 and HOXA9 genes is indicative of the presence of lung cancer in said human. In another embodiment, a hypermethylation status of the SCT, SHOX2 and HOXA7 genes is indicative of the presence of lung cancer in said human.

The present disclosure also provides the use of the methylation status of the CpG site in at least one gene selected from HOXA9, SHOX2, SCT and HOXA7 as a biomarker for lung cancer detection.

The present disclosure also provides a composition for detecting or diagnosing lung cancer in a human, comprising at least two of the primer pairs mentioned above. The composition may further comprise the corresponding probes.

### BRIEF DESCRIPTION OF THE FIGURES

Embodiments of the invention are herein described, by way of non-limiting example, with reference to the following accompanying figures:

FIGS. 1A and 1B depict the nucleotide sequence (A) and ROC curve (B) of HOXA 7, with the methylation sites shaded grey and the positions of PCR primers and probe underlined.

FIGS. 2A and 2B depict the nucleotide sequence (A) and ROC curve (B) of SCT, with the methylation sites shaded grey and the positions of various PCR primers and probes underlined.

FIGS. 3A and 3B depict the nucleotide sequence (A) and ROC curve (B) of SHOX2, with the methylation sites shaded grey and the positions of PCR primers and probe underlined.

FIGS. 4A and 4B depict the nucleotide sequence (A) and ROC curve (B) of HOXA9, with the methylation sites shaded grey and the positions of PCR primers and probe underlined.

### DETAILED DESCRIPTION OF THE INVENTION

### Definitions

Various terms used throughout this specification shall have the definitions set forth herein.

As used herein, the term “CpG” island refers to a genomic region that contains a high frequency of CpG sites. “C” and “G” refer to cytosine and guanine, respectively, while “p” refers to the phosphodiester bond between the cytosine and the guanine, which indicates that the C and the G are next to each other in sequence. A CpG island is characterized by CpG dinucleotide content of at least 60% of that which would be statistically expected (about 4-6%), whereas the rest of the genome has a much lower CpG frequency (about 1%).

As used herein, the term “methylation site” when used in the context of a CpG island refers to a site where a C is immediately followed by a G that is present on a CpG island.

As used herein, the term “genomic DNA” refers to DNAs found within the 46 chromosomes in humans. The genomic DNA provides a complete set of genetic information including coding and non-coding DNA.

As used herein, the term “sodium bisulfite” refers to sodium hydrogen sulfite having the chemical formula of NaHSO₃. Sodium bisulfite functions to deaminate cytosine into uracil; but does not affect 5-methylcytosine (a methylated form of cytosine with a methyl group attached to carbon 5). When the bisulfite-treated DNA is amplified via polymerase chain reaction, the uracil is amplified as thymine and the methylated cytosine is amplified as cytosine.

While bisulfite convention coupled sequencing is regarded as a gold-standard technology for detection of DNA methylation because it provides a qualitative, quantitative and efficient approach to identify 5-methylcytosine at single base-pair resolution, one of ordinary skills in the art will readily recognize that several other methods are available, including Methylation Specific PCR (MSP), Combined Bisulfite Restriction Analysis (COBRA), Methylation-sensitive Single Nucleotide Primer Extension (Ms-SNuPE) and several other techniques depending on different applications (Rand K et al., (2002) Conversion-specific detection of DNA methylation using real-time polymerase chain reaction (ConLight-MSP) to avoid false positives. Methods. 27:114-120; Xiong and Laird, (1997) COBRA: a sensitive and quantitative DNA methylation assay. Nucleic Acids Res. 25:2532-2534; Gonzalgo and Jones, (1997) Rapid quantitation of methylation differences at specific sites using methylationsensitive single nucleotide primer extension (Ms-SNuPE) Nucleic Acids Res. 25:2529-2531; and Suzuki and Bird, (2008) DNA methylation landscapes: provocative insights from epigenomics. Nat Rev Genet. 9:465-476).

Furthermore, other methods are available for converting C to U such as using enzymes such as NEBNext Enzymatic Methyl-seq, and methylation specific PCR described above can also be used to detect hypermethylation of these genes.

As used herein, the term “methylation” refers to the addition of a methyl group to the 5′ carbon of the cytosine base in a deoxyribonucleic acid sequence of CpG within a gene on a human chromosome.

As used herein, the term “methylation status” refers to the presence or absence of a methylated cytosine base at a methylation site in a CpG island within a gene. Methylation of a CpG island is often associated with inhibition of gene expression. A CpG island often begins upstream of a gene in the promoter. For purposes of this application, a CpG island can span the promoter region, the coding region (e.g., exons), and the non-coding region (e.g., introns) of a gene.

As used herein, the term “methylation specific PCR” refers to the use of primer pairs in a PCR reaction that are complimentary to DNA that is converted by sodium bisulfite and that contains several CpG dinucleotides (i.e., multiple methylation sites) that can be methylated in vivo. Primers can be complimentary to the methylated template where methylated CpG cytosines are not converted to uracil by the sodium bisulfite treatment and non-CpG cytosines are converted to uracil.

As used herein, the term “real time PCR” (also called quantitative real time polymerase chain reaction) refers to a method for the detection and quantitation of an amplified PCR product based on incorporation of a fluorescent reporter dye; the fluorescent signal increases in direct proportion to the amount of PCR product produced and is monitored at each cycle, “in real time”, such that the time point at which the first significant increase in the amount of PCR product correlates with the initial amount of target template.

As used herein, the term “primer set” refers to a pair of PCR primers that include a forward primer and reverse primer used in a PCR reaction and allows the generation of an amplicon.

As used herein, the term “probe” refers to a TaqMan probe used in a real time PCR and it consists of a fluorophore covalently attached to the 5′-end of an oligonucleotide designed such that it anneals within a DNA region amplified by a specific set of primers and a quencher at the 3′-end. The quencher molecule quenches the fluorescence emitted by the fluorophore when excited by a light source via FRET (Fluorescence Resonance Energy Transfer). As the Taq polymerase extends the primer during PCR and synthesizes the nascent strand the 5′ to 3′ exonuclease activity of the polymerase degrades the probe that has annealed to the template. Degradation of the probe releases the fluorophore from it and breaks the close proximity to the quencher, thus relieving the quenching effect and allowing fluorescence of the fluorophore.

As used herein, the term “detecting the methylation status of a gene” refers to detecting or assessing the presence or absence of a methylated cytosine base in a CpG island within a gene of interest. Methods of detecting the methylation status of a gene include, for example, nested methylation specific PCR, methylation specific PCR or bisulfite sequencing.

As used herein, the term “HOXA9” refers to human homeoboxA9 which is a member of the homeobox cluster A. The nucleotide sequence is deposited in a GenBank database with NCBI Reference Sequence: NM_152739, the disclosure of which is incorporated herein by reference. As used herein, the term “SHOX2” refers to human short statue homeobox 2 which is a member of the homeobox family. The nucleotide sequence is deposited in a GenBank database with NCBI Reference Sequence: NM_006884, the disclosure of which is incorporated herein by reference. As used herein, the term “SCT” refers to human secretin which is glucagon family of peptides. The nucleotide sequence is deposited in a GenBank database with NCBI Reference Sequence: NM_021920, the disclosure of which is incorporated herein by reference. HOXA7″ refers to human homeoboxA7 which is a member of the homeobox cluster A. The nucleotide sequence is deposited in a GenBank database with NCBI Reference Sequence: NM_006896, the disclosure of which is incorporated herein by reference. Using publically available genomic sequence databases (e.g., genome browser provided by UCSC, http://genome.ucsc.edu/index.html), the location of the CpG island for a particular gene can be determined. For example, the CpG island for SCT and SHOX2 may be nucleotides 388-846 of SEQ ID NO.: 17 and nucleotides 216-618 of SEQ ID NO.:18, respectively.

It is known in the field that there are many methylation sites that are present on a CpG island with respect to a particular gene. A methylation site is characterized by a cytosine (C) immediately followed by a guanine (G) that are present within a CpG island. It is also recognized by one skilled in the art that a majority of methylation sites (present within a CpG island of a particular gene) are unmethylated in healthy cells. During cancer development, these methylation sites within a CpG island undergo DNA methylation.

The present invention provides a method of detecting DNA methylation (i.e., detecting DNA methylation status) in bronchial washing or bronchial brush of a plurality of genes including HOXA7, SCT, SHOX2 and HOXA9. An increase in DNA methylation of these genes is found to be correlated with lung cancer.

The present invention provides obtaining bronchial washing from patients suspected of having lung cancer. Saline was used for bronchial washing. About 15-30 ml of saline was injected into the lung closest to potential lung cancer lesion and then was retrieved.

In one embodiment, freshly collected samples may be used for DNA methylation assay. In another embodiment, collected samples may be frozen immediately and stored at a preferred temperature range of −20° to −80° C.

The present invention provides for isolating the genomic DNA from the sample. In one embodiment, the washing may be briefly centrifuged at 3,000 rpm for 15 minutes. Supernatant was removed. PBS was used to wash the cell pellets and centrifuged at 3,000 rpm for 15 minutes. Supernatant was removed and cell pellets can be stored at −80° C.

One skilled in the art would recognize numerous DNA extraction protocols. In one embodiment, DNA is extracted using a mixture of phenol and chloroform. Preferably, the mixture contains 50% phenol, 48% chloroform, and 2% isoamyl alcohol. Commercially available purification kits (for genomic DNA isolation) may also be used. During the DNA extraction procedure, degraded protein contaminants as well as cell debris are removed.

To detect DNA methylation of a particular region of genomic DNA associated with a gene that contains a CpG island, the present method provides for first converting the isolated genomic DNA so that the majority of unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. Preferably, isolated DNA is treated with sodium bisulfite (NaHSO₃) which converts unmethylated cytosine to uracil, while methylated cytosines are maintained (Hayatsu H. et al., (1970) Reaction of sodium bisulfite with uracil, cytosine and their derivatives. Biochemistry 9, 2858-2865).

The nucleotide conversion results in a change in the sequence of the original DNA. It is general knowledge that the resulting uracil has the base pairing behavior of thymine, which differs from cytosine base pairing behavior. To that end, uracil is recognized as a thymine by Taq polymerase. Therefore after PCR, the resultant product contains cytosine only at the position where 5-methylcytosine occurs in the starting template DNA. This makes the discrimination between unmethylated and methylated cytosine possible. Useful conventional techniques of molecular biology and nucleic acid chemistry for assessing sequence differences are well known in the art (See, for example, Sambrook, J., et al., (2001) Molecular cloning: A laboratory Manual, 3rd edition, Cold Spring Harbor, N.Y.).

The present inventors developed an optimal cancer detection using DNA methylation. Although DNA methylation is sensitive and specific, it may be difficult to utilize this test to detect lung cancer in bronchial washing samples. This is so because there is a limited number of tumor cells present in the sample. It is hypothesized, that because of the disproportionate high numbers of healthy cells relative to low numbers of tumor cells, that a highly sensitive DNA methylation assay is required. This is so especially during the early stage of lung cancers. If the selected CpG island present in a gene is methylated in a few cancer cells, any methylation activity from the thousands of healthy cells may affect the background signals (i.e., high background noise).

It remains a difficult task to select a panel of genes that undergo DNA methylation and is restricted to lung cancer patients. The present inventors selected a novel panel of genes containing HOXA7, SHOX2, SCT and HOXA9 that represents an improvement over the prior art. The present invention provides detection of DNA methylation using this panel of genes and the DNA methylation assay unexpectedly has a high sensitivity and specificity for detecting lung cancer cells.

The present invention provides a method of detecting DNA methylation using a plurality of specific genes. It is discovered that DNA methylation of SCT and HOXA7 sufficiently provides a high sensitivity of >74% and a specificity of >84%. Also, it is discovered that DNA methylation of SHOX2 and HOXA7 sufficiently provides a high sensitivity of >74% and a specificity of >78%. It is further discovered that DNA methylation of SHOX2 (in addition to SCT and HOXA7) further increases its specificity to >91%. It is further discovered that DNA methylation of HOXA9 (in addition to SCT and HOXA7) further increases its specificity to >88%.

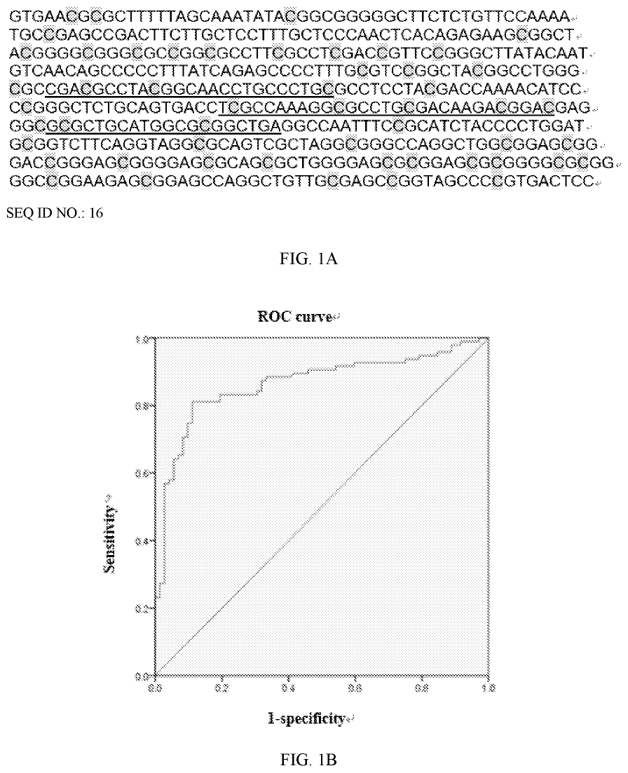

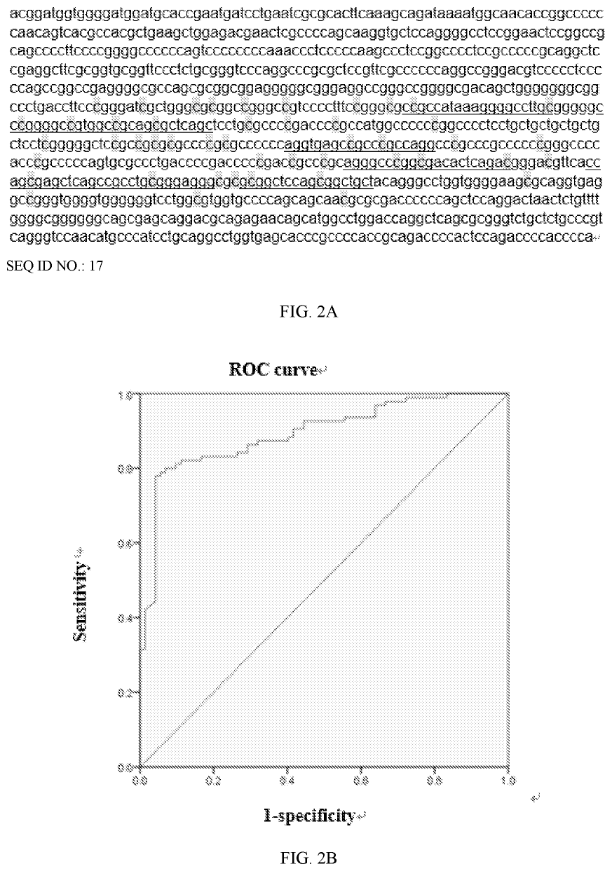

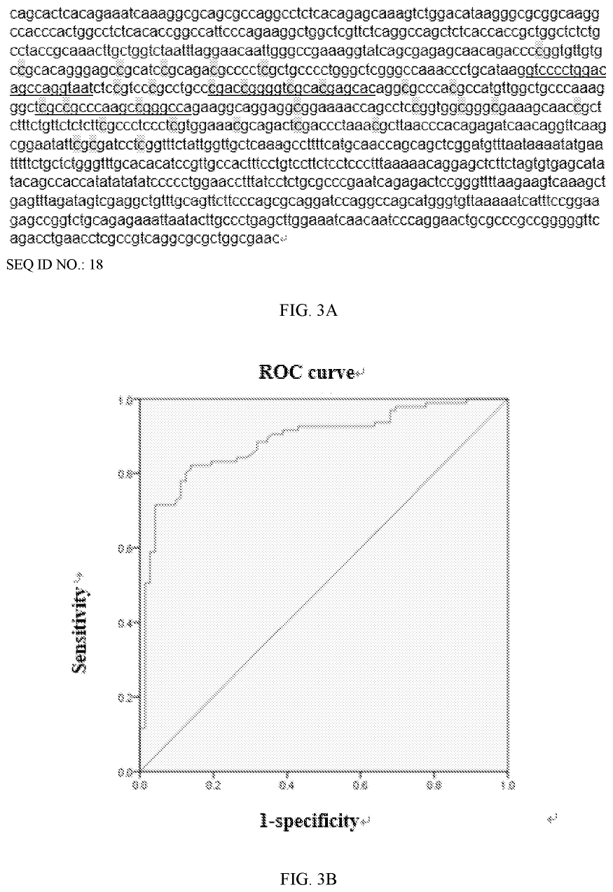

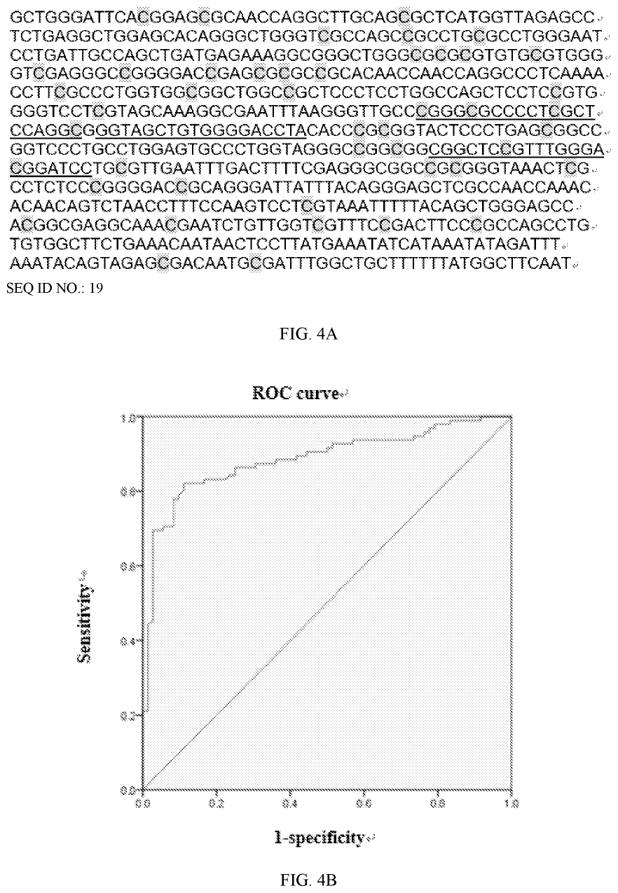

The present invention discloses that detection of methylation in any of the specific CpG site of the CpG island depicted in FIG. 1-4 is sufficient as an biomarker for lung cancer. Thus, in addition to using the specific primers disclosed herein below for amplifying the relevant segment of the genes, other PCR primers can be devised, or other methods used to detect the methylation of these sites.

In one embodiment, the present invention provides a method of using bisulfite sequencing to identify “real” methylation sites present on a CpG island (i.e., methylated CpG only in cancer cells but not in healthy cells). The protocol of bisulfite sequencing is known by one skilled in the art. In short, isolated DNA is treated with sodium bisulfite followed by sequencing or PCR reaction to provide information relating to the locations of methylated CpG dinucleotides present within a CpG island. Primer sets for sequencing or PCR reaction with respect to the four genes can be designed.

PCR primers are selected based, in part, on the following criteria. First, the size of the PCR products (i.e., amplicon) is in the range of about 80-350 base pairs.

The PCR primers must be bisulfite specific, i.e. they must hybridize to cytosines. These cytosines are followed immediately by guanine (i.e., CpG cytosines). In other words, if these cytosines are not be methylated, they will always be converted to uracil after bisulfite treatment. Primers that are not bisulfite specific (i.e., hybridize to less than 4 non-CpG cytosines) may hybridize to DNA that are not fully converted by sodium bisulfite and thus resulting in false positive results.

The PCR primers should avoid (i) a consecutive of >3 cytosines, or (ii) a consecutive of >3 cytosines and thymines in combination. Such consecutive cytosines or cytosines/thymines will both be recognized as thymines after cytosines are converted to uracils following bisulfite treatment. In the event that there are >3 consecutive cytosines or cytosines/thymines, it would force the PCR primers to contain a consecutive >3 adenines. These PCR primers would suffer from (i) a low annealing temperature, (ii) a decrease in specificity (i.e., non-specific PCR).

In accordance with the present invention, PCR primers should have a sufficiently high annealing temperature so that any annealing of mismatch of the primers to a non-specific sequence of DNA would not occur. Thus, a sufficiently high annealing temperature would avoid production of a non-specific amplicon.

The present invention provides primer sets for each of the four (4) selected genes (i.e., HOXA7, SHOX2, SCT and HOXA9). In one embodiment, PCR primer sets include:

HOXA7, SEQ ID NO: 1 (forward primer) and SEQ ID NO: 2 (reverse primer);
SCT (set 1), SEQ ID NO: 4 (forward primer) and SEQ ID NO: 5 (reverse primer), or alternatively SCT (set 2), SEQ ID NO: 7 (forward primer) and SEQ ID NO: 8 (reverse primer);
SHOX2, SEQ ID NO: 10 (forward primer) and SEQ ID NO: 11 (reverse primer); and
HOXA9, SEQ ID NO: 13 (forward primer) and SEQ ID NO: 14 (reverse primer).

As discussed above, other methylation detection techniques may be used for the present invention. One of ordinary skilled in the art recognizes that there are many existing techniques for determining methylation status of genes. For example, methylation assays include, but are not limited to, sequencing, methylation-specific PCR (MS-PCR), melting curve methylation-specific PCR, MLPA with or without bisulphite treatment, MSRE-PCR (Melnikov et al, (2005) MSRE-PCR for analysis of gene-specific DNA methylation. Nucleic Acids. Res. 33(10):e93), bisulphite conversion-specific methylation-specific PCR (BS-MSP) (Sasaki et al., (2003) Bisulfite conversion-specific and methylation-specific PCR: a sensitive technique for accurate evaluation of CpG methylation. Biochem Biophys Res Commun. 309(2):305-9), and the like. A review of techniques for determining DNA methylation analysis is provided in DAMHA M J AND NORONHA A, (1998) Recognition of nucleic acid double helices by homopyrimidine 2′,5′-linked RNA Nucleic, Acids Research, Vol. 26, No. 10, 2255-2264, and P. W. Laird, (2003) The power and the promise of DNA methylation markers. Nature Reviews Cancer, Vol. 3, no. 4, 253-266, the disclosure of which are incorporated herein in their entirety.

In one aspect, the present invention provides the use of methylated human genomic DNA as controls, which provides confirmatory information regarding the presence of sufficient genomic DNA in the sample (e.g., >10 ng). Such information is essential for the interpretation of the methylation status of genes in the present invention. If there is control amplification of methylated human genomic DNA (Ct<305 from the PCR assay), it indicates a sufficient amount of genomic DNA present in the sample. If there is no control amplification (Ct>35 from the PCR assay), it indicates that there is insufficient amount of genomic DNA. In the present invention, methylated genomic DNAs were used as controls, using the same primers and probes as those for the particular genes.

The present methylation assay can detect methylation status of genes using less than 20 ml of washing from patients. The methylation status from the present assay correlates with the biopsy confirmation of lung tumors. The present methylation assay has a high sensitivity and specificity. The present assay detects no methylation in healthy lung, indicating specificity.

The following examples are provided to further illustrate various preferred embodiments and techniques of the invention. It should be understood, however, that these examples do not limit the scope of the invention described in the claims. Many variations and modifications are intended to be encompassed within the spirit and scope of the invention.

### EXAMPLES

### Materials and Methods

Bronchial washing was collected through bronchoscopy. About 15-30 ml of saline was injected into the lung closest to potential lung cancer lesion and then was retrieved. Centrifugation at 3,000 rpm for 15 minutes was used to separate cell pellets and supernatant. PBS was used to wash the cell pellets and centrifuged at 3,000 rpm for 15 minutes. Supernatant was removed and cell pellets were used for DNA extraction.

DNAs were extracted from bronchial washing and bronchoscopic brushes. Bisulfite treatment was used for the conversion of unmethylated cytosine to uracil. PCR was used to determine the methylation status of HOXA7, HOXA9, SCT, and/or SHOX2, using methylated human genomic DNAs (Cat #57821,CpGenome) as controls. The methylation sites and sequences of these four genes are shown in FIG. 1-4.

In specific, PCR was performed using Taqman Universal PCT master mix (Cat #4324018, Applied Biosystem), following the instructions of the manufacturer, and primers and probes used for PCR were as follows.

| | HOXA7 forward primer: |
|---|---|
| (SEQ ID NO.: 1) | |
| | CGACGTTTACGGTAATTTGTTTTGC |
| | HOXA7 reverse primer: |
| (SEQ ID NO.: 2) | |
| | TCAACCGCGCCATACAACG |
| | HOXA7 probe: |
| (SEQ ID NO.: 3) | |
| | TCGTTAAAGGCGTTTGCGATAAGACGGAC |
| | SCT forward primer1: |
| (SEQ ID NO.: 4) | |
| | AGGGTTCGGCGATATTTAGAC |
| | SCT reverse primer1: |
| (SEQ ID NO.: 5) | |
| | AACAACCGCTAAAACCGC |
| | SCT probe1: |
| (SEQ ID NO.: 6) | |
| | CCCTCCCGCAAACGACTAAACTCGCTAA |
| | SCT forward primer2: |
| (SEQ ID NO.: 7) | |
| | TCGTTATAAAGGGGTTTTGC |
| | SCT reverse primer2: |
| (SEQ ID NO.: 8) | |
| | CCTAACGAACGACTCACCT |
| | SCT probe2: |
| (SEQ ID NO.: 9) | |
| | TTCGGGGTCGTGGTCGTAGCGTTTAGT |
| | SHOX2 forward primer: |
| (SEQ ID NO.: 10) | |
| | GTTTTTTGGATAGTTAGGTAAT |
| | SHOX2 reverse primer: |
| (SEQ ID NO.: 11) | |
| | TAACCCGACTTAAACGACGA |
| | SHOX2 probe: |
| (SEQ ID NO.: 12) | |
| | CTCGTACGACCCCGATCG |
| | HOXA9 forward primer: |
| (SEQ ID NO.: 13) | |
| | CGGGCGTTTTTCGTTTTAGGC |
| | HOXA9 reverse primer: |
| (SEQ ID NO.: 14) | |
| | AAATCCGTCCCAAACGAAACCG |
| | HOXA9 probe: |
| (SEQ ID NO.: 15) | |
| | TAAATCCCCACAACTACC |

### Results

A genome-wide 850K Illumina methylation array analysis was performed using bronchial washing from patients with lung cancer and benign lung diseases. It has been found that the methylation status of the following genes can distinguish lung cancer from benign lung diseases, as they showed differential methylation levels between lung cancer samples and benign lung disease samples: NFAM1, BZRAP1, MIR142, C160RF68, MIR365-1, SPNS1, LAT, SIX1, MAB21L1, MIR548, NBEA, BIN2, ALX1, CUX2, C11ORF21, TSPAN32, WDFY4, TRIM39, LOC728264, MIR145, HOXA9, STAB1, ARHGAP25, PRDM16, SCT, NDUFS2, FCER1G, SHOX2, ETVS, DPP10, PADI6, BAIAP2L1, MEIS1, DLGAP2, WDR66, JAM3, RBM24, RBM38, HOXD8, GBX2, DPP6, KCNIP4, SHANK2, CBLN1, SRGAP3, PCDHGA4, PCDHGC3, PCDHGA12, PCDHGA11, PCDHGA9, PCDHGA1, PCDHGB1, PCDHGCS, PCDHGB6, PCDHGB3, PCDHGB7, PCDHGA6, PCDHGA8, PCDHGA10, PCDHGAS, PCDHGB4, PCDHGC4, PCDHGA3, PCDHGA2, PCDHGA7, PCDHGB2, PCDHGCS, PCDHGBS, PCDHGC3, PCDHGB19P, MYCBPAP, HOXA7, FRR3, SPATA16, DRDS, PTCH1, SCN10A, C2CD4D, LOC100132111, YBX2, ELAVL4, GPR146, ZNF135, TFAP2E, HCN1, CMTM2, SLC12A7, TRIM58, LHX1, KCNC2, FRAT1, ESPN, RPH3AL, EN1, FAM135B, SCARF2, SLC32A1, PTCH1, LHX8, TBX1, FAT1, ARHGEF4, ZNF781, GPSM1, DUOX1, DLG4, ALX3, SYK, SLC32A1, SLC43A2, ASAM, EXOC3, SIX2, CDH4, CKB, IRX4, ZNF135, SNCB, HOXD10, RPH3AL, ISL2, HOXD3, CACNG8, ZNF529, LINC00554, GPR88, C2CD2, ZNF667, CRB2, WDR8, PRDM14, TAC1, NLFN1, MRI1, HOXD9, SRCIN1, GPR177, SLC38A1, DLXS, GPR88, CDH13, AKR7L, C4ORF39, TRIM61, PACRG, GPR6, FLJ45983, ZNF582, HLA-A, LAMA4, RGS12, HSF2BP, ZNF544, SDK1, ZNF382, MTUS1, CD52, UBXN11, LOC101929563.

Four of these candidate genes, HOXA7, HOXA9, SCT, SHOX2, were validated in larger sample size. Bronchial washing fluids were collected from 122 patients who underwent bronchoscopy examination. Among these samples, 53 were from lung cancer patients, and 69 were from patients with benign lung diseases. DNAs were extracted from these samples and bisulfate-treated, the methylation status of these four genes was measured by PCR to determine whether these genes can distinguish lung cancer from benign lung diseases. The hypermethylation of these four genes were found in samples from cancer patients.

It has been found that each gene was able to distinguish cancer from benign lung diseases: HOXA9 sensitivity 63%, specificity 76.8%; SHOX2 sensitivity 59.3%, specificity 82.6%; SCT sensitivity 61.1%, specificity 91.3%; HOXA7 sensitivity 59.3%, specificity 92.8% (Table 1). The analysis using the combination of any two or three or four of these biomarkers showed that the combinations yielded better sensitivity and specificity. For example, the combination of HOXA7 and SCT had the sensitivity 74.1%, specificity 84.1% (Table 2); the combination of HOXA7, SCT and SHOX2 had the sensitivity 68.5%, specificity 91.3% (Table 3). The combination of four biomarkers had the sensitivity 74.1%, specificity 88.4% (Table 1). The ROC curves of the four genes were created by plotting sensitivity against (1-specificity) and shown in FIG. 1-4.

It was also tested on whether the SCT CpG island (not just the fragment amplified by PCR) being detected can serve as a biomarker for lung cancer detection. Two pairs of primer and probe were designed on the same CpG island and then tested in bronchial washing. It was found that these two pairs of primers showed similar results on lung cancer detection (Table 5). These results showed that this CpG island can serve as a biomarker for lung cancer detection.

Also bronchoscopic brushes were collected from 167 patients to determine whether these brushes can be used for lung cancer diagnosis, 97 of 167 samples from lung cancer patients and the other 70 from patients with benign lung diseases. These brushes were used for cytopathology analysis first, then DNAs were extracted from the brushes. It was found that each of these genes was able to distinguish lung cancer from benign lung diseases: HOXA9 sensitivity 82.1%, specificity 88.9%; SHOX2 sensitivity 82.1%, specificity 86.1%; SCT sensitivity 77.9%, specificity 95.8%; HOXA7 sensitivity 81.1%, specificity 88.9% (Table 4). The combinations of two biomarkers yield better sensitivity and specificity. For example, the combination of HOXA9 and SCT had the sensitivity 90%, specificity 91.5% (Table 4), the combination of SCT and HOXA7 also had the sensitivity 90%, specificity 91.5% (Table 4).

TABLE 1
Specificity and sensitivity of each biomarker in bronchoalveolar lavage

| | HOXA9 | SHOX2 | SCT* | HOXA7 | all_combined |
|---|---|---|---|---|---|
| Sensitivity (%) | 62.96 | 59.26 | 61.11 | 59.26 | 74.07 |
| Specificity(%) | 76.81 | 82.61 | 91.3 | 92.75 | 88.41 |
| Youden index | 0.398 | 0.419 | 0.524 | 0.52 | 0.625 |
| AUC | 0.699 | 0.709 | 0.762 | 0.760 | 0.851 |
| 95% CI | 0.603~0.794 | 0.614~0.804 | 0.672~0.852 | 0.669~0.851 | 0.782~0.927 |
| P | <0.001 | <0.001 | <0.001 | <0.001 | <0.001 |

*SCT forward primer1, SCT reverse primer1 and SCT probe1 were used.

TABLE 2
The result of the combined analysis of two biomarkers. 1: HOXA9; 2:
SHOX2; 3: SCT*; 4: HOXA7

| | 1 and 2 | 1 and 3 | 1 and 4 | 2 and 3 | 2 and 4 | 3 and 4 |
|---|---|---|---|---|---|---|
| Sensitivity (%) | 79.63 | 61.11 | 77.78 | 61.11 | 74.07 | 74.07 |
| Specificity(%) | 63.77 | 91.30 | 75.36 | 91.30 | 78.26 | 84.06 |
| AUC | 0.779 | 0.785 | 0.809 | 0.791 | 0.807 | 0.828 |
| P | <0.001 | <0.001 | <0.001 | <0.001 | <0.001 | <0.001 |

*SCT forward primer1, SCT reverse primer1 and SCT probe1 were used.

TABLE 3
The result of the combined analysis of three biomarkers.
1: HOXA9; 2: SHOX2; 3: SCT*; 4: HOXA7

| | 1, 2 and 3 | 1, 2 and 4 | 1, 3 and 4 | 2, 3 and 4 |
|---|---|---|---|---|
| Sensitivity (%) | 61.11 | 64.81 | 70.37 | 68.52 |
| Specificity(%) | 95.65 | 91.30 | 88.41 | 91.30 |
| AUC | 0.805 | 0.838 | 0.839 | 0.839 |
| P | <0.001 | <0.001 | <0.001 | <0.001 |

*SCT forward primer1, SCT reverse primer1 and SCT probe1 were used.

TABLE 4
Specificity and sensitivity of each biomarker in bronchoscopic brush

| | | | | | HOXA9 and |
|---|---|---|---|---|---|
| | HOXA9 | SHOX2 | SCT* | HOXA7 | SCT* |
| Sensitivity (%) | 78.4 | 67.6 | 81.1 | 75.7 | 86.4 |
| Specificity(%) | 95.8 | 100 | 95.8 | 87.5 | 100 |

*SCT forward primer1, SCT reverse primer1 and SCT probe1 were used.

TABLE 5
Specificity and sensitivity of SCT in bronchial washing using two
different pairs of primer and probe on the same CpG island

| | SCT1 | SCT2 |
|---|---|---|
| Sensitivity(%) | 53.7 | 61.11 |
| Specificity(%) | 94.2 | 91.3 |
| AUC | 0.740 | 0.762 |
| P | <0.001 | <0.001 |

Nondiagnostic bronchoscopic examination is an unmet clinical need. It was then examined whether these biomarkers can improve bronchoscopic diagnosis. In the 15 cases where bronchoscopic examination was nondiagnostic and lung cancer was ultimately diagnosed, 12 were correctly diagnosed using our assay (combination of SCT, HOXA7 and SHOX2) in bronchial washing. Sensitivity was 80% in nondiagnostic populations. In combination with cytopathology, the sensitivity reached 94.4% in patients who underwent broncoscopic examination. These results demonstrated that the test significantly improved bronchoscopic diagnosis.

In summary, it was demonstrated that the methylation status of HOXA7, HOXA9, SCT, and/or SHOX2 in bronchial washing and bronchoscopic brush can be used to improve the diagnostic performance of bronchoscopy in lung cancer.

### Discussion

Nondiagnostic bronchoscopic examination is a major problem in lung cancer diagnosis. Two major factors cause bronchoscopic nondiagnosis: first bronchoscopy is not able to reach all lesions especially peripheral lesions even with navigation system; second navigation system does not guide bronchoscopy to lesions accurately which causes biopsy site is not lesion site. Bronchial washing is able to reach all lung lesions. The assay mentioned above also showed high specificity and sensitivity in lung cancer detection by bronchoscopy. In nondiagnostic bronchoscopic cases where lung cancer was ultimately diagnosed, the assay reduced nondiagnosis rate, significantly improved bronchoscopic diagnosis.

Although it has been shown many genes are hypermethylated in lung cancer tissue when compared to normal human lung tissue, it was not known that the combination of a limited number of gene hypermethylation can be used for lung cancer diagnosis especially in bronchial washing and brush.

HOXA7, HOXA9, SCT, SHOX2 individually has been shown to be hypermethylated in lung cancer tissue. However, individual hypermethylation of any of these four genes is not sufficient for lung cancer diagnosis.

It could not have been assumed that the combination of these genes can improve performance just because they individually show efficacy.

The results above showed that the combination of these four genes or the combination of three or two of these genes can be used for lung cancer diagnosis in bronchial washing and bronchial brush.

To our knowledge, the assay above showed highest specificity and sensitivity than all the commercially available assays for lung cancer diagnosis and differentiation of malignant lung nodule from benign lung nodule.

1. A method of diagnosing lung cancer in a human, comprising the steps of:
(a) isolating genomic DNA from a bronchial washing sample or bronchial brush from a human, wherein a plurality of genes consisting of HOXA9, SHOX2, SCT and HOXA7 are present in said genomic DNA;
(b) converting unmethylated cytosine to uracil in the genomic DNA using sodium bisulfite;
(c) performing a round PCR on said sodium bisulfite-treated genomic DNA to amplify, said round PCR is performed using a forward primer and a reverse primer for SCT and HOXA7 respectively, said forward primers consisting of the nucleotide sequence of SEQ ID NO:1 (for HOXA7) and SEQ ID NO: 4 (for SCT), and said reverse primers consisting of the nucleotide sequence of SEQ ID NO:2 (for HOXA7) and SEQ ID NO: 5 (for SCT); and
(d) detecting the presence or absence of PCR amplification result;
wherein the presence of a PCR amplification result in one of the genes indicates the presence of a hypermethylation status of the gene, and wherein the presence of a hypermethylation status of both of the genes is indicative of the presence of lung cancer in said human.

2. The method of claim 1, wherein in step c), the round of PCR is performed on said sodium bisulfite-treated genomic DNA using a forward primer and a reverse primer for SHOX2, SCT and HOXA7 respectively, said forward primers consisting of the nucleotide sequence of SEQ ID NO:1 (for HOXA7), SEQ ID NO: 4 (for SCT) and SEQ ID NO: 10 (for SHOX2), and said reverse primers consisting of the nucleotide sequence of SEQ ID NO:2 (for HOXA7), SEQ ID NO: 5 (for SCT) and SEQ ID NO: 11 (for SHOX2); and
wherein the presence of a PCR amplification result in one of the genes indicates the presence of a hypermethylation status of the gene, and wherein the presence of a hypermethylation status of all of the genes is indicative of the presence of lung cancer in said human.

3. The method of claim 1, wherein in step c), the round of PCR is performed on said sodium bisulfite-treated genomic DNA using a forward primer and a reverse primer for HOXA9, SCT and HOXA7 respectively, said forward primers consisting of the nucleotide sequence of SEQ ID NO:1 (for HOXA7), SEQ ID NO: 4 (for SCT) and SEQ ID NO: 13 (for HOXA9), and said reverse primers consisting of the nucleotide sequence of SEQ ID NO:2 (for HOXA7), SEQ ID NO: 5 (for SCT) and SEQ ID NO: 14 (for HOXA9); and
wherein the presence of a PCR amplification result in one of the genes indicates the presence of a hypermethylation status of the gene, and wherein the presence of a hypermethylation status of all of the genes is indicative of the presence of lung cancer in said human.

4. A method of diagnosing lung cancer in a human, comprising the steps of:
(a) isolating genomic DNA from a bronchial washing sample or bronchial brush from a human, wherein a plurality of genes consisting of HOXA9, SHOX2, SCT and HOXA7 are present in said genomic DNA;
(b) converting unmethylated cytosine to uracil in the genomic DNA using sodium bisulfite;
(c) performing a round PCR on said sodium bisulfite-treated genomic DNA to amplify, said round PCR is performed
i) using a forward primer and a reverse primer for HOXA9 and HOXA7 respectively, said forward primers consisting of the nucleotide sequence of SEQ ID NO:1 (for HOXA7) and SEQ ID NO: 13 (for HOXA9), and said reverse primers consisting of the nucleotide sequence of SEQ ID NO:2 (for HOXA7), and SEQ ID NO: 14 (for HOXA9),
ii) using a forward primer and a reverse primer for SHOX2 and HOXA7 respectively, said forward primers consisting of the nucleotide sequence of SEQ ID NO:1 (for HOXA7) and SEQ ID NO: 10 (for SHOX2), and said reverse primers consisting of the nucleotide sequence of SEQ ID NO:2 (for HOXA7) and SEQ ID NO: 11 (for SHOX2), or
iii) using a forward primer and a reverse primer for HOXA9, SHOX2, and SCT respectively, said forward primers consisting of the nucleotide sequence of SEQ ID NO:4 (for SCT), SEQ ID NO:10 (for SHOX2) and SEQ ID NO:13 (for HOXA9), said reverse primers consisting of the nucleotide sequence of SEQ ID NO:5 (for SCT), SEQ ID NO:11 (for SHOX2) and SEQ ID NO:14 (for HOXA9); and
(d) detecting the presence or absence of PCR amplification result;
wherein the presence of a PCR amplification result in one of the genes indicates the presence of a hypermethylation status of the gene, and wherein the presence of a hypermethylation status of both or all of the genes is indicative of the presence of lung cancer in said human.

5-6. (canceled)

7. A method of diagnosing lung cancer in a human, comprising the steps of:
(a) isolating genomic DNA from a bronchial washing sample or bronchial brush from a human,
wherein a plurality of genes consisting of HOXA9, SHOX2, SCT and HOXA7 are present in said genomic DNA;
wherein the HOXA7 gene comprises a sequence of SEQ ID NO: 16, the SCT gene comprises a sequence of SEQ ID NO: 17, the SHOX2 gene comprises a sequence of SEQ ID NO: 18, and the HOXA9 gene comprises a sequence of SEQ ID NO: 19, and
wherein each of said gene comprises at least one CpG island which may be methylated,
(b) converting unmethylated cytosine to uracil in the genomic DNA using sodium bisulfite;
(c) performing a round PCR on said sodium bisulfite-treated genomic DNA to amplify, wherein said round PCR is performed to determine if said CpG island in said gene is methylated,
and wherein the presence of a hypermethylation status of at least two of the genes is indicative of the presence of lung cancer in said human.

8. The method of claim 7, wherein a hypermethylation status of at least three of the genes is indicative of the presence of lung cancer in said human.

9. The method of claim 8, wherein a hypermethylation status of the HOXA7 and SCT genes is indicative of the presence of lung cancer in said human.

10. The method of claim 9, wherein a hypermethylation status of the SHOX2, HOXA7 and SCT genes is indicative of the presence of lung cancer in said human.

11. The method of claim 8, wherein a hypermethylation status of the HOXA9, HOXA7 and SCT genes is indicative of the presence of lung cancer in said human.

12. The method of claim 8, wherein a hypermethylation status of the SHOX2 and SCT genes is indicative of the presence of lung cancer in said human.

13. The method of claim 8, wherein a hypermethylation status of the SHOX2 and HOXA7 genes is indicative of the presence of lung cancer in said human.
